(19) United States
(12) Patent Application Publication
(10) Pub. No.: US 2021/0374572 A1
(43) Pub. Date: Dec. 2, 2021

(54) Inferring the Impact on a User's Well-Being of Being in a Certain Location or Situation or with Certain Individuals

(71) Applicant: Koa Health B.V., Barcelona (ES)

(72) Inventors: Tiago Manuel de Simas, Sintra (PT); Jesus Alberto Omaña Iglesias, Barcelona (ES)

(21) Appl. No.: 17/330,345

(22) Filed: May 25, 2021

(30) Foreign Application Priority Data
May 28, 2020 (EP) 20382454.5

(51) Int. Cl. G06N 5/04; G06F 9/451; G16H 50/30

(52) U.S. Cl. CPC G06N 5/04; G16H 50/30; G06F 9/451

(57) ABSTRACT

A method for efficiently extracting user data to improve the user's well-being involves acquiring user data through the user's interactions with an application running on the user's smartphone. Based on the user data, a graphical model is generated with nodes whose magnitudes correspond to values of the user data. A recommended range of values of user data is determined based on state vectors and diffusion metrics derived from the graphical model. The user data is a list of pairs of values of current state personal constructs X and desired state opposite personal constructs Y. The graphical model includes a bipartite graph indicative of how frequently specific pairs of personal and opposite personal constructs (X,Y) appear in the list. The recommended range of values ranks activities that the application recommends to the user to improve the user's well-being by enabling the user to reach the desired state from the current state.

### CROSS REFERENCE TO RELATED APPLICATION

This application is based on and hereby claims the benefit under 35 U.S.C. § 119 from European Patent Application No. EP 20382454.5, filed on May 28, 2020, in the European Patent Office. This application is a continuation-in-part of European Patent Application No. EP 20382454.5, the contents of which are incorporated herein by reference.

### TECHNICAL FIELD

The present invention has its application within the telecommunications sector and, more specifically, relates to the deployment of tools using electronic devices and communication electronic devices (e.g., mobile user terminals such as smartphones, tablets, wearable electronic devices, computers, etc.) that measure and send a user's parameters and/or interact with the user in order to estimate and improve his/her well-being (for example, alleviating stress). More particularly, the present invention relates to a method and electronic system for an efficient extraction of a user's well-being data and recommendations (similar to what personal construct theory does face-to-face with a psychiatrist), which allows accurate recommendations to be derived in order to improve the user's well-being.

### BACKGROUND

Since 2008, the leading causes of death in the United States have resulted from personal choices and behavior, and the same trend is seen in many other countries worldwide. In order to be able to change behavior that damages the health of an individual, the personal constructs of such individuals on certain topics must first be understood. In other words, it must be understood how an individual reacts to certain events and circumstances. In this regard, repertory grids exist within the personal construct theory, which is an interview technique (developed by George Kelly) that has proven to be successful in inferring from an individual the ways in which he or she extracts meaning from the observations and events that occur to him/her or around him/her.

The repertory grid is an interviewing technique that uses non-parametric factor analysis to determine a measure of personality. In other words, the repertory grid is a technique for identifying the ways that a person construes, interprets or gives meaning to his or her experience. It provides information from which inferences about personality can be made. But it is not a personality test in the conventional sense.

The repertory grids are based on the personal construct theory developed by George Kelly in which he describes humans as “incipient scientists” who create hypotheses in order to understand and interpret events. In this context, the repertory grid is a technique designed to explore the structure and content of these hypotheses. Mathematical techniques that extract meaning from repertory grids have been developed because an interesting feature of the repertory grid technique is that the results can be analyzed mathematically. Some approaches have been proposed in this regard, as for example shown in: E. Faccio, M. Castiglioni & R. C. Bell, “Extracting information from repertory grid data: New perspectives on clinical and assessment practice,” TPM—Testing, Psychometrics, Methodology in Applied Psychology, Vol. 19, No. 3, September 2012, 177-196.

Repertory grid techniques have been applied in different domains, for example in clinical, subclinical and marketing settings, among others. For instance, repertory grid techniques have been used for assessing work-related stress and for assessing whether a given treatment would be effective.

Repertory grid interviews are usually performed between a psychiatrist and a patient (face to face), and typically it takes around one hour from start to finish, thus making it not feasible to be implemented at scale. Generally speaking, the repertory grid techniques as currently applied, have some common requirements and characteristics that render them inefficient at saving resources and being applied quickly and at large scale (applicable to a very large number of users). These requirements are:

Location: The interview must be performed in an office (or any other predefined location), i.e., it cannot be taken in whatever place the user wants.

Scale: A psychiatrist must perform the interview face-to-face with the user.

Time: Typical repertory grid interviews last between 45 minutes to more than one hour; the reason is mainly that the psychiatrist will attempt as much as possible to explore the answers from the users, given that it is not convenient to bring the user to the predetermined location (e.g., the psychiatrist office) multiple times.

No-anonymity: The individual (the user or, in other words, the person giving the repertory grid interview) must feel comfortable telling his/her psychiatrist his/her personal views on a given topic. Thus, feelings of judgment and shame might not allow the user to express himself/herself as honestly as he or she would like to.

Therefore, a need exists for a method and system for applying repertory grids to users in an accurate, personalized and resource saving manner that allows accurate recommendations for behavior modification to be derived in order to improve the user's well-being by avoiding undesired behavior in the future.

### SUMMARY

A method is disclosed for efficiently extracting repertory grid data and for providing recommendations for improving the well-being of a patient. A novel system provides a way to collect user data from the interactions of the patient with an application running on an electronic device. The application enables repertory grids and personal constructs from one or more users to be extracted in such a way that the users need not explicitly enter or provide the repertory grid data. This structured way of collecting data allows individual personal constructs to be extracted in a massively scalable fashion with minimum resources. Additionally, the extracted data is recurrently input into a novel recommender system or engine that results in greater engagement efficacy for the user, helping the patient with his or her decision-making process.

A method for efficiently extracting repertory grid data and other user data to improve a well-being state of a user involves acquiring the user data through interactions of the user with an application running on the user's electronic device and receiving the user data onto a server. The user data relates to interactions that occurred over a first period of time. Based on the user data, a graphical model for the user is generated that has nodes whose magnitudes correspond to values of the user data. The graphical model is updated based on new user data acquired during a later period of time through the application running on the electronic device of the user.

A recommended range of the values of the user data is determined based on state vectors and diffusion metrics derived from the graphical model for the user. The user data is a list of pairs of values of a current state personal construct X and an desired state opposite personal construct Y. The graphical model for the user includes a bipartite graph indicative of how frequently specific pairs of personal constructs and opposite personal constructs (X,Y) appear in the list.

The graphical model includes an extended graph that includes a projection of the bipartite graph to the personal construct nodes X and a projection of the bipartite graph to the opposite personal construct nodes Y. The projections are generated using a similarity measure, such as a fuzzy Jaccard similarity measure. The values rank activities that the application recommends to the user in order to improve the user's well-being. The recommended range of values ranks the activities to be undertaken by the user according to the likelihood that the user will reach the desired state from the current state by engaging in each activity. The application recommends the ranked activities to the user through a user interface of the electronic device of the user.

A system for efficiently extracting user data and improving the well-being state of a user includes a database and an electronic device with a processing unit. The electronic device can be a laptop, a tablet, a personal computer, a portable computer, a mobile phone, a smartphone or a smartwatch. The database stores user data that is acquired through interactions of the user with an application running on the electronic device of the user. The user data relates to interactions that occurred over a first period of time. The processing unit of the electronic device of the user is adapted to execute program instructions of the application that, when executed, cause the electronic device to retrieve data, generate a graphical model, determine recommended values and recommend an activity. The graphical model is updated based on new user data acquired during a later period of time through the application running on the electronic device of the user.

The user data is retrieved from the database. The graphical model for the user is generated based on the user data and has nodes whose magnitudes correspond to values of the user data. The user data is a temporal ordered list of pairs of values of a personal construct X and an opposite personal construct Y of the user corresponding to a time period, such as the first period of time or the later period of time. The personal construct X represents a current state of the user, whereas the opposite personal construct Y represents a desired state of the user. The graphical model for the user includes a bipartite graph indicative of how frequently specific pairs of personal constructs and opposite personal constructs (X,Y) appear in the list. The graphical model for the user has an extended graph that includes a projection of the bipartite graph to the personal construct nodes X and a projection of the bipartite graph to the opposite personal construct nodes Y. A recommended range of the values of the user data is determined based on state vectors and diffusion metrics derived from the graphical model for the user. The values rank activities that the application recommends to the user. The application recommends that the user engage in one of the ranked activities.

Other embodiments and advantages are described in the detailed description below. This summary does not purport to define the invention. The invention is defined by the claims.

### BRIEF DESCRIPTION OF THE DRAWING

The accompanying drawings, where like numerals indicate like components, illustrate embodiments of the invention.

FIG. 1 shows an schematic example on how a mobile application (called REMIX) can be used to extract repertory grids data.

FIG. 2 shows a schematic example of how the extended graph is built from the bipartite graph (BG), according to an embodiment of the present invention.

FIG. 3 is a table of user data collected from a mobile app over a period of time.

FIG. 4 is a bipartite graph of the variables X and Y using the data of the table of FIG. 3.

FIG. 5 is an adjacency matrix of the graph projection XX based on the bipartite graph of FIG. 4.

FIG. 6 is an adjacency matrix of the graph projection YY based on the bipartite graph of FIG. 4.

FIG. 7 is a table showing the adjacency matrix ZXY of the z-score calculation of the projection XY.

FIG. 8 is a table showing the adjacency matrix ZXX of the z-score calculation of the projection XX.

FIG. 9 is a table showing the adjacency matrix ZYY of the z-score calculation of the projection YY.

FIG. 10 is a table of nodes and weights used to generate a scaled and normalized extended bipartite graph.

FIG. 11 is a table showing an n-diffusion ordered vector obtained from a user of a mobile app.

### DETAILED DESCRIPTION

Reference will now be made in detail to some embodiments of the invention, examples of which are illustrated in the accompanying drawings.

The problems found in prior art techniques are generally solved or circumvented, and technical advantages are generally achieved, by the disclosed embodiments which provide a method and system for efficient application of repertory grid techniques and well-being improvement recommendations. The proposed method and system provide a novel way to collect users' data from user application interactions (in electronic devices) by enabling repertory grids (also called repertory grid data) and personal constructs from one or more users to be extracted in such a way that the users need not explicitly fill the repertory grid data. Hence, it can be said that the repertory grids are extracted in a passive way without requiring the user actively to provide the repertory grids, for example, by answering specific questions. Thus, the extracted repertory grids can be called Passive Digital Repertory Grids (PDRG).

Extracting information using PDRG is a structured way of collecting data that allows users' Personal Constructs to be inferred and is a way psychologically to characterize an individual. This allows the user's psychological state to be assessed, which can be modeled and fed to a recommender engine with the goal of helping the individual's well-being to be improved.

The design of some electronic device applications (for example, applications aimed at reducing stress or any other type of application) are leveraged in order to extract passive digital repertory grids (PDRGs) such that individual (user) personal constructs can be understood and such that the user does not have to explicitly provide the repertory grid data. That is, the PDRGs are acquired based on how the user interacts with an electronic device application (e.g., a smartphone application), which has the advantage of constantly and passively (from the user's point of view) acquiring the repertory grids for the user, in a similar way as a specialist would acquire through a personal interview. The repertory grid technique has so far not been integrated into any mobile or smartphone application in such a way that the user does not explicitly fill out the repertory grid.

One of the advantages of the proposed solution (when compared with the problems of the current repertory grid techniques as previously stated) is that it allows Individual Personal Constructs to be extracted in a massively scalable fashion with minimum resources. Therefore, the novel method will allow group characterizations and comparisons to be created between many users (individuals), which is only possible when reaching a large scale of users.

Additionally, PDRGs are recurrently input into a novel recommender system or engine (RecSys), which results in higher engagement efficacy for the user and helps the individual in his/her decision-making process. The novel system feeds the personal constructs of the users as input into the recommender system. In one embodiment, the recommender system uses state vectors and diffusion metrics to propose activities that will be easier (i.e., activities that create a smooth transition from the current to the desired state of the user) for the user to engage in (measured, for instance, through click rate) and to obtain better results (i.e., increase well-being and reduce stress).

In a first aspect, it is proposed a computer implemented method efficiently extracts personal user data and improves the well-being state of the users. The method includes the following steps performed by one or more electronic processors of an electronic system:

a) receiving and storing in a database, data related to one or more users, in which the data related to a user is acquired through interactions of the user with an application running in an electronic device of the user via a user interface of the electronic device;

b) generating a graphical model for at least one user (from the one or more users whose data has been stored in the database) by processing a set of data related to the user, obtained from the database (data acquired through interactions of the user with an application running in an electronic device of the user) and corresponding to a certain period of time, wherein nodes of the graphical model correspond to the different values of the set of data;

c) for each user for which a graphical model has been generated, determining a recommended range of values for parameters or activities related to the user (which usually should result in an improvement of the well-being score of the user) based at least on the generated graphical model for the user, and using state vectors and diffusion metrics obtained from the graphical model generated for the user.

The state vector may be perceived from the application running in the electronic device that captures data over time (e.g., in the form t, current state variable, future state variable), for example by the user stating how he/she feels and how he/she wants to feel in the future.

The application used to acquire the set of data related to the user (through user interactions with the application) may be many software applications, as for example, a user's well-being application such as REMIX or any other user's well-being application.

The database may be a database external to the electronic devices of the one or more users (that is, a database stored in an electronic device external to the user's electronic devices), and the electronic devices of the one or more users send the acquired data through a communications network to the external device that stores the database, such as a remote server.

In an alternative embodiment, as the graphical model is generated per user, the novel system has the ability to run without having to store data in a server, but instead uses the data stored in the user electronic device.

The one or more electronic processors performing the method (and the database) can be located in an electronic device of one of the users. Alternatively, the one or more processors and the database can be located in one or more remote servers external to the user's electronic devices. The one or more user's electronic devices that acquire the data related to the users send the acquired data through a communications network to the remote server(s).

The electronic device of each user is, for example, a laptop, a tablet, a personal computer, a portable computer, a mobile phone, a smartphone or a smartwatch.

In one embodiment, step b) above further includes updating the graphical model based on a new set of data acquired through the application running in the electronic device of the user. The new set of data corresponds to a different later period of time (later than the certain period of time). Step b) also includes updating the generated graphical model for a user every time new data for the user, which is acquired after the certain period of time through user interactions with the application running in the user's electronic device, is received and stored in the database.

In one embodiment, the set of data related to a user is a temporal ordered list of pairs of values of a personal construct X, and an opposite personal construct Y, of the user corresponding to the certain period of time. The graphical model for a user includes a graph (a Bipartite Graph) that is based on the frequency that a specific pair of values of personal constructs and opposite personal constructs (X,Y) appear in the list. The weights of the edges in the graphical model are, for example, given by the frequency that a specific pair of personal constructs and opposite personal constructs (X,Y) appear in the list. The graphical model for the user can include an extended graph, which includes the Bipartite Graph. The projections of the Bipartite Graph to the personal construct nodes X and the projections of the Bipartite Graph to the opposite personal construct nodes Y are built using a similarity measure, such as the Fuzzy Jaccard Similarity measure. The extended graph may also include the transpose of the Bipartite Graph.

Optionally, to build the extended graph, the scale effect between the Bipartite Graph and the projections is reduced, for example, by using a z-score technique.

The personal construct and the opposite personal construct may be a current state and a desired state of the user.

In one embodiment, step c) above includes the substeps:

c1) obtaining an initial state and a target well-being goal for a user;

c2) based on the graphical model generated for the user and on the initial state and the target goal, applying an n-diffusion closure distance to determine the shortest diffusion path between the initial state and the target goal;

c3) based on the determined shortest diffusion path, determining a recommended range of values for parameters and activities related to the user in order to achieve the target goal from the initial state of the user.

The determined recommendation for a user (recommended range of values for parameters or activities related to the user) is presented to the user through a user interface of the electronic device of the user.

A second aspect of the invention involves a system that performs the method of the first aspect. For example, the second aspect involves an electronic system for efficiently extracting personal user data and for improving the well-being state of users. The system includes:
a means for receiving and storing in a database data related to one or more users that is acquired through interactions of the user with an application running on an electronic device of the user; and
at least one processor configured to:
   obtain from the database a set of data (related to at least one user) corresponding to a certain period of time;
   generate a graphical model for the at least one user by processing the set of data related to the user that corresponds to the certain period of time, where nodes of the graphical model correspond to different values of the set of data; and
   for each user for which a graphical model has been generated, determine a recommended range of values for parameters or activities related to the user based at least on the generated graphical model for that user, using state vectors and diffusion metrics obtained from the graphical model generated for the user.

A third aspect of the invention involves a computer program product including computer program code adapted to perform the method of the first aspect. The computer program code is executed on a computer, a digital signal processor, a field-programmable gate array, an application-specific integrated circuit, a micro-processor, a micro-controller, or any other form of programmable hardware. A non-transitory digital data storage medium is also provided for storing a computer program that includes instructions that cause a computer executing the program to perform the method of the first aspect.

The embodiments of the invention can be implemented in a variety of architectural platforms, operating and server systems, devices, systems, or applications. Any particular architectural layout or implementation presented herein is provided for purposes of illustration and comprehension only and is not intended to limit aspects of the invention.

The proposed embodiments provide a method and system for efficiently extracting personal constructs of the users and for providing well-being improvement recommendations (e.g., by feeding the extracted user's construct to a recommender engine) for modifying a user's damaging behavior.

In order to understand and change a user's behavior, it is fundamental to attain a better understanding of how the human mind works. The human brain and mind are multiscale adaptive self-organizing complex systems. the brain and mind can be modeled as a set of complex associations or complex networks. By hypothesis, behavior relies on an associative set of memories that are built-up through individual interaction with the environment and on the replaying of the associative memory network. The mind can be seen as an adaptive self-organizing multiscale network of memories distributed in a manifold that folds space-time perception of the user.

The novel system approaches the substrate acquisition in order subsequently to exploit the human behavior dynamics in space-time. In that regard, the repertory grids approach is used.

Specifically, a grid includes four parts or types of components:
a topic: concerning some part of the user's experience.
a set of elements (1 . . . k) which are examples or instances of the topic: the elements can, for example, be some activities undertaken to reach a particular state related to the topic, or some objects related to the topic.
a set of constructs (1 . . . n): the constructs can, for example, be current and desired states (related to the topic) of the user or generally speaking basic terms that the client uses to make sense of the elements. The constructs were formerly expressed as a contrast.
a set of ratings (of elements on constructs): feedback from the user about the elements (for example about the recommended activity). For example, each element can be positioned between the two extremes of the construct using a rating scale system. This is performed repeatedly for all the constructs that apply.

An example of a repertory grid about a topic, in short, is the relation between elements and constructs. For the topic “addiction”, examples of elements are: to consume heroin, cocaine, alcohol, etc., which would be rated by the topic's constructs and opposite construct, such as: (feel the buzz, not feel the buzz), (feel sexy, not feel sexy), (makes me sick, makes me healthy), (feel happy, feel sad), etc.

In general, the repertory grids technique currently consists of personal interviews about a given topic with the strategy of first eliciting from or priming the interviewed user with a set of initial questions that follows with the free elicitation by the user that defines the set of elements and constructs and its relative association value. Repertory grids are snapshots of this memory multi-scale adaptive self-organized associative complex system of an individual in space-time. The interview relates to how the person in question positions or values a set of elements relative to each other in the space-time of the interview. Most decision making varies in space-time, i.e., depends on the context and value gain or cost reduction of the decision to be taken towards the goal.

However, the conventional way of acquiring repertory grids lacks spatial and temporal resolution. The novel method solves this problem by using Passive Digital Repertory Grids (PDRG). PDRG is different than conventional repertory grids because PDRG repertory grids are used to obtain information about several topics continuously over space-time through an electronic device application. One problem facing conventional repertory grids is that users are frequently unwilling to constantly answer questionnaires, so they cannot be continuously interviewed. The novel method overcomes this problem by extracting several repertory grids (i.e., elements, constructs and ratings) based on the interactions of the users with the electronic device application, such as a mobile applications.

In one embodiment, the electronic device applications are applications adapted to increase the well-being of the user, such as REMIX. In this specific app, the user responds about his current emotional state or well-being and the state (emotional/well-being) that he wants to achieve, which operates as a dual construct. The recommended activities operate as the elements of the repertory grid. The rating of each recommended activity, rates or values the elements with respect to the construct. Over time, the app acquires the repertory grid of the user being studied with good spatial and temporal resolution. Moreover, this type of approach can be scaled to a massive cohort of users.

The user's electronic devices running applications used to extract the repertory grids may be any one of the following: a laptop, a tablet, a personal computer, a portable computer, a mobile phone, a smartphone, a smartwatch, or any other electronic device with which the user can interact and on which an application can run.

The invention does not depend on any particular type of electronic device or application. Many types of applications can be used, so long as the application is able to capture the PDRG repertory grids data and, preferably, store the data in a consistent (remote or local) database.

The data captured by the electronic device application over time regarding a specific user is stored in a database. The database may be internal to one of the user's electronic devices or may be external to the user's electronic devices (for example, in a remote server). The electronic device has a communication interface that allows the electronic device to communicate the acquired data to the database (directly or through another electronic device) over a communications network. The communications network may be a wireless communications network, a mobile communications network such as GSM 2G, GPRS, UMTS, 3G, 4G, LTE, LAN or W-LAN or any other type of wired or wireless communication networks. For simplicity, it is supposed that all the captured data is stored in a single database. But generally speaking, the data can be stored in one or more databases in the same or different electronic devices.

The logic of the application stores, for each topic, constructs (e.g., current and desired states from an user) that may represent a personal construct and the opposite personal construct, elements (e.g., activities to reach a particular state), and ratings (e.g., feedback from the user about the recommended activity).

In another embodiment, the novel method based on repertory grids acquisition allows an individual's decision making to be modeled as an interacting agent-based system. The agent is defined as the dynamic weighted network (based on repertory grids) that values the associations between elements and constructs. With this network of associations at a given space-time, it is possible to estimate the individual value function regarding the elements and constructs and to allow the most probable outcome of a possible decision to be predicted by the weighted network agent.

A use case is now described in which a mobile application (in this case the REMIX application, also known as REMIX) is used. The application logic (way of working), the model for extracting the repertory grids, and the recommender system are now described. This is only a non-limiting example, and many other electronic device applications can be used to implement the novel method.

Application Logic:

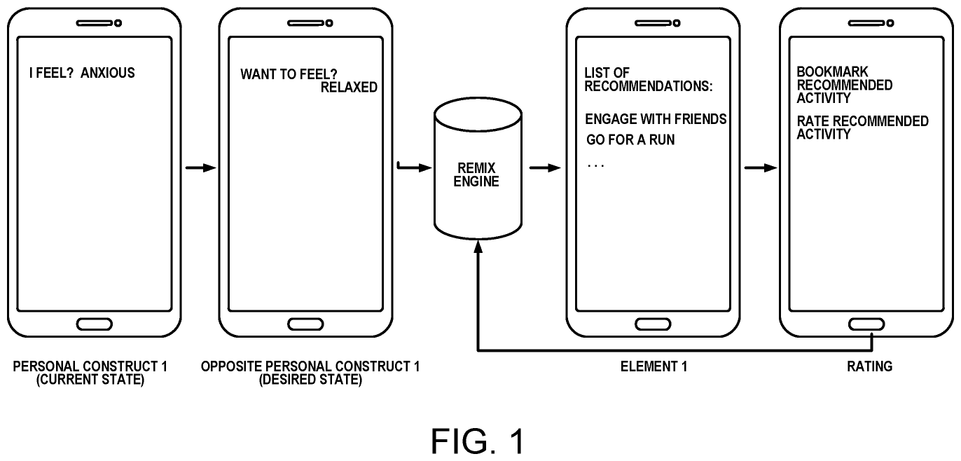

FIG. 1 shows a schematic example of how a mobile application (named REMIX) can be used for repertory grids extraction. FIG. 1 also shows the Logic Flow of digital repertory grids as implemented by the REMIX app. The user is asked at time t to provide information about “how he/she feels” (personal construct 1, current state) and “how he/she wants to feel” (opposite personal construct 1, desired state). The goal of the app is to provide the best match activity as a recommendation from a set of possible activities that will help the user achieve the goal “how he/she wants to feel”. In order to do that, the intelligence of the mobile app (the REMIX engine) recommends a list of activities (element 1) which are rated by the user.

For example, in the past the user acquired an “item A1” at time t, and an “item A2” at time t+1. The task of the recommender system is to prime the user to choose an item A3 based on this past information or to select a ranked set of items {A, B, C, D, . . . ). The mobile app REMIX categorizes “how he/she feels” as “item A1” and “how he/she wants to feel” as “item A2”. The mobile app acquires data as a temporal list of pairs “how he/she feels” and “how he/she wants to feel” at times t1, t2, . . . , tn. In one embodiment, a data engine of the mobile app builds a graphical model for each user from this information. For example, a graph is built where the nodes are the various components of the repertory grids, and the graph edges (lines) are the various unions or relations (preferably weighted) between those components. The graph evolves over time as the user interacts with the mobile app. The graphical model is baseline or input of the recommender system.

The user information that is extracted from the electronic device application at each point in time is interpreted as different components of the repertory grids of the user at different times. A graphical model or personal graph the evolves over time is built with the components of the extracted repertory grid for the user and the inter-relations between the components. The graphical model or personal graph is fed to the recommender system to generate the appropriated recommendations.

Model:

One non-limiting way to build the graphical model is now described. However, other ways of building the model can be used. A temporal ordered list of pairs of personal constructs and opposite personal constructs is generated for each user using the user interactions with the electronic device application. For example, a pair of construct and opposing construct is denoted LU=[t, “how he/she feels”, “how he/she wants to feel” ], where t is the time to which each construct corresponds.

The temporal ordered list of pairs is represented as a bipartite graph BG=[{X,Y},E{x,y}], where X and Y are the set of node labels in the set of possible constructs X and opposite constructs Y (“how he/she feels” X and “how he/she wants to feel ‘Y’) respectively. E{x,y} is the set of weighted edges that connects node x with node y. In one embodiment, the weights of the edges correspond to the frequency observed in the LU list (the frequency that a particular X corresponds to a particular Y, X=>Y) for each user. Both the LU list and the bipartite graph BG expand over time with more user interactions with the electronic device through the mobile application. The following node labels are used by the REMIX mobile app: array([‘bored’, ‘stressed’, ‘annoyed’, ‘worried’, ‘angry’, ‘anxious’, ‘relaxed’, ‘sleepy’, ‘sad’, ‘excited’, ‘miserable’, ‘content’, ‘calm’, ‘happy’, ‘adventurous’, ‘creative’, ‘inspired’, ‘ashamed’, ‘excited’, ‘calm’, ‘happy’, ‘productive’, ‘inspired’, ‘sleepy’, ‘creative’, ‘content’, ‘relaxed’, ‘andventerous’], dtype=object)

The extended bipartite graph BG is generated for each user based on an adjacency matrix and on projections of the two states “how he/she feels” and “how he/she wants to feel”. This extended graph represents the graphical model for each user. How the extended graph is generated is described in more detail below.

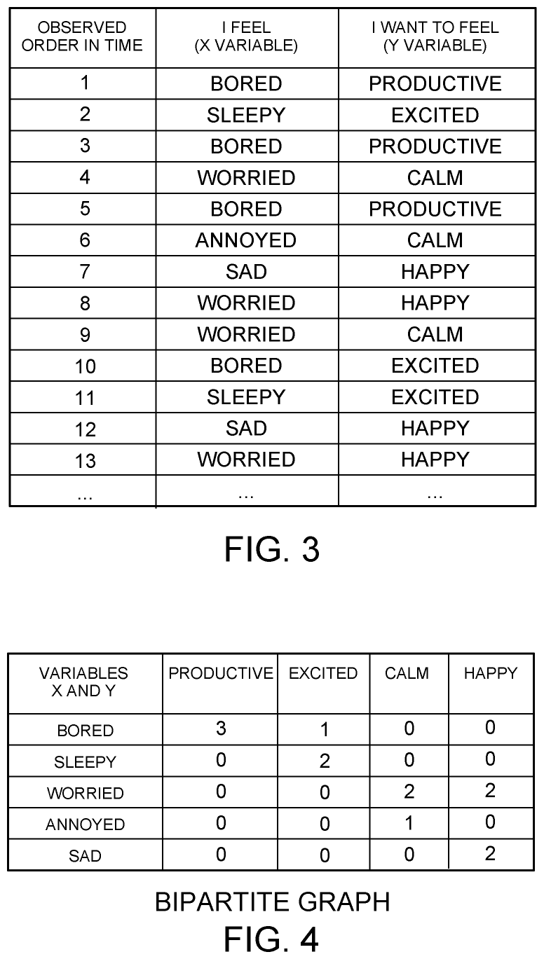

1. The table of FIG. 3 represents an example of data collected from the electronic device application, such as REMIX, over a period of time (for example, a week or any other period of time).

2. A bipartite graph is generated using the table of FIG. 3, where the nodes are the variables (X,Y), and the edges are the frequency at which a particular X corresponds to a particular Y (X=>Y) at any specific time t. The table of FIG. 4 is the bipartite graph BG generated with the data (X,Y) from the table of FIG. 3 for the time period up through time t=13. Entries of the table of FIG. 4 represent the number of times that a particular X corresponds to a particular Y (X=>Y) in the table of FIG. 3.

3. The projections of XY into XX and YY are generated from the entries in the table of FIG. 4. Any similarity measure can be employed to generate these projections. In one embodiment, the Fuzzy Jaccard Similarity measure with (max,min) is used because it has the property of creating semi-metric space, defined as:

$${J}_{( i , j )} = \frac{{\sum }_{k = 1}^{n} min ( {v}_{i , k} , {v}_{j , k} )}{{\sum }_{k = 1}^{n} max ( {v}_{i , k} , {v}_{j , k} )} \qquad ( Equation 1 )$$

where v_{i,k} denotes the element k of vector i.

In the specific example described above, for the projection of XY into XX, the following vectors are used (the rows of the table of FIG. 4):

X₁=(3,1,0,0)

X₂=(0,2,0,0)

X₃=(0,0,2,2)

X₄=(0,0,1,0)

X₅=(0,0,0,2)

Then to obtain the projection of XY into XX, the fuzzy Jaccard measure of all possible pairs J(Xᵢ,Xⱼ)=J(X_{i,j}) is calculated according to equation 1 as follows:

J(X_{1, 1})=[min (3,3)+min (1,1)+min (0,0)+min (0,0)]/ [max(3,3)+max(1,1)+max(0,0)+max(0,0)]=(3+1+0+0)/ (3+1+0+0)=4/4=1.0

J(X_{1,2})=[min (3,0)+min (1,2) +min (0,0) +min (0,0)]/ [max(3,0)+max(1,2)+max(0,0)+max(0,0)]=(0+1+0+0)/(3+2+0+0)=1/5=0.2

. . .

J(X_{2,4})=[min (0,0)+min(2,0)+min (0,0)+min (0,2)]/ [max(0,0)+max(2,0)+max(0,0)+max(0,2)]=(0+0+0+0)/(0+2+0+2) =0/4=0.0

. . .

J(X_{5,5})=[min(0,0)+min(0,0)+min(0,0)+min(2,2)]/ [max(0,0)+max(0,0)+max(0,0)+max(2,2)]=(0+0+0+2)/ (0+0+0+2)=2/2=1

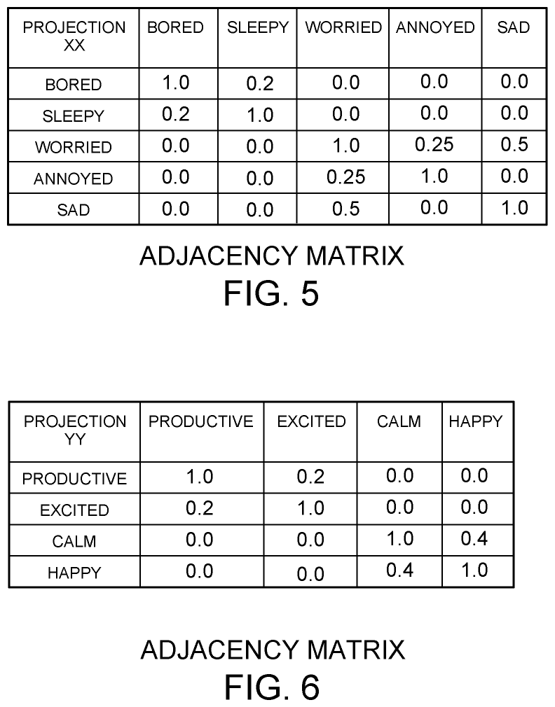

The table of FIG. 5 shows the final result for t=13 of the projection XX, which is an adjacency matrix of the graph projection XX.

Continuing with the specific example of the tables of FIGS. 3-4, for the projection of XY into YY, the following vectors are used (the columns of the table of FIG. 4):

Y₁=(3,0,0,0,0)

Y₂=(1,2,0,0,0)

Y₃=(0,0,2,1,0)

Y₄=(0,0,2,0,2)

Therefore, according to equation 1:

J(Y_{1,1})=[min (3,3)+min(0,0)+min (0,0)+min (0,0) +min(0,0)]/[max(3,3)+max(0,0)+max(0,0)+max(0,0)+max(0,0)]=(3+0+0+0+0)/(3+0+0+0+0)=3/3=1.0

J(Y_{1,2})=[min (3,1)+min (0,2)+min (0,0)+min (0,0) +min(0,0)]/[max(3,1)+max(0,2)+max(0,0)+max(0,0)+max(0,0)+max(0,0)]=(1+0+0+0+0)/(3+2+0+0+0)=1/5=0.2

. . .

J(Y_{2,4})=[min (1,0)+min(2,0)+min (0,2)+min(0,0)+min (0,0) +min(0,2)]/[max(1,0)+max(2,0)+max(0,2)+max(0,0)+max(0,2)]=(0+0+0+0+0)/(1+2+2+0+2)=0/7=0.0

. . .

J(Y_{4,4})=[min(0,0)+min(0,0)+min(2,2)+min(0,0) +min(2,2)]/[max(0,0)+max(0,0)+max(2,2)+max(0,0)+max(2,2)]=(0+0+2+0+2)/(0+0+2+0+2)=4/4=1.0

The table of FIG. 6 shows the final result for t=13 of the projection YY, which is an adjacency matrix of the graph projection YY.

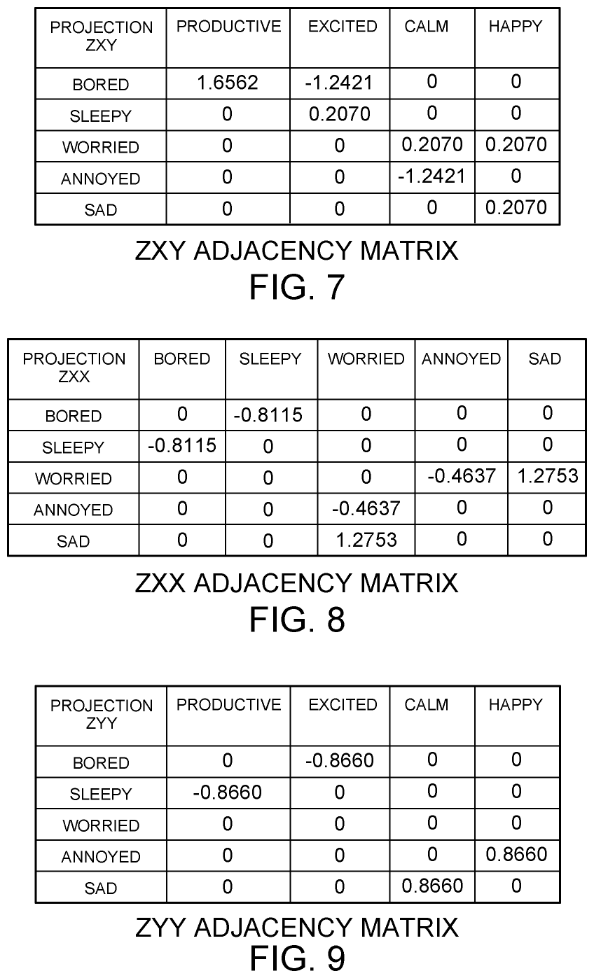

4. In one embodiment, scale effects between XY, XX and YY are removed. This is done because, in general, XY, XX and YY are in different scales, and the scale effects should be removed or at least reduced. In order to reduce the scale effects, a z-score may be used. Here, XY is z-scored to obtain the matrix ZXY considering for the calculation only the non-zero elements of XY, i.e., for the z-score calculation all zeros from table of FIG. 4 are excluded. Similarly, all zero elements of XX and YY are also excluded. The z-score calculations of XX and YY are based on the tables of FIGS. 5-6 by removing from the calculation the zero values as well as the diagonal elements in this case. The adjacency matrixes ZXY, ZXX and ZYY are obtained, as shown in the tables of FIGS. 7, 8 and 9, respectively.

The z-scored Extended Graph of XY is then generated from the adjacency matrixes ZXY, ZXX and ZYY of FIGS. 7-9:

$$Z {E}_{B G} = [ \begin{matrix}Z X X & Z X Y \\ Z X {Y}^{T} & ZYY\end{matrix} ]$$

5. Normalized Extended Graph: Next, in one embodiment, the z-scored Extended Graph ZEB_{G} is normalized by removing all non-zero entries as well as the diagonal elements. The z-scored Extended Graph is normalized into the unit interval [0,1] by means of a unique linear function that maps W:[a,b]=>[0,1]:

$${w}_{i j} = \frac{( 1 - 2 ɛ ) {Z}_{i , j} + ( 2 ɛ - 1 ) min ( Z )}{max ( Z ) - min ( Z )} + ɛ \qquad ( Equation 2 )$$

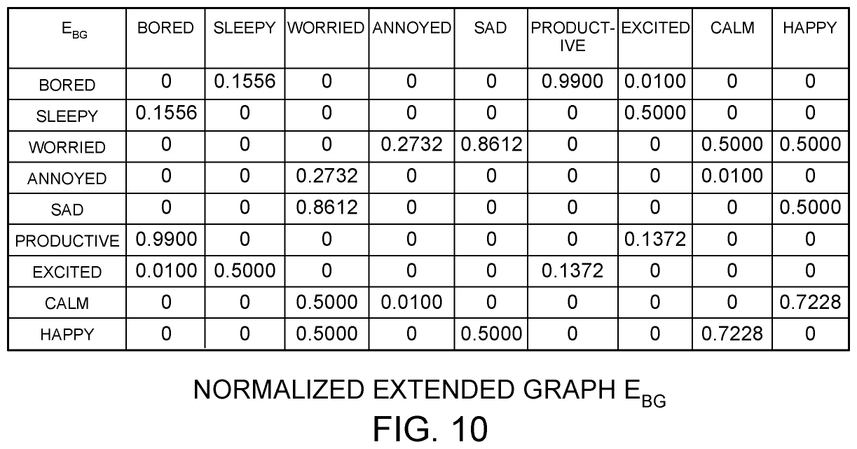

The scaled and normalized extended bipartite graph E_{BG} is obtained with this process using equation 2, for example, with ε=0.01. The extended graph is generated using the nodes and weights of the table of FIG. 10.

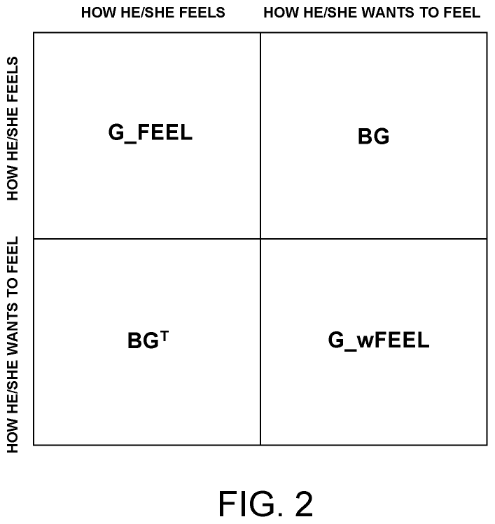

FIG. 2 shows a schematic example of how the extended graph is built from the bipartite graph (BG). In FIG. 2, BG is the original bipartite graph generated as previously explained. G_Feel is a projection of the bipartite graph to the personal construct nodes X, current state (“How he/she feels”). G_wFeel is a projection of the bipartite graph to the opposite personal construct nodes Y, desired state (“How he/she wants to feel”). BG^{T} is the transpose of the bipartite graph BG. In all four corners of FIG. 2, the scale effect is removed or reduced, for example, as explained in Simas et al., “An algebraic topological method for multimodal brain networks comparisons,” Frontiers in Psychology, Jul. 6, 2015, Vol. 6, Article 904 (DOI:10.3389/fpsyg.2015.00904).

In summary, the information observed from the user is the bipartite graph BG as described above. G_Feel and G_wFeel are obtained by projecting BG information on X and Y, respectively. These projections are generated using similarity measures such as the Jaccard similarity measure, cosine, etc. In general, these four corners of the extended graph are embedded in different scales, and the scale effects can be removed using different known procedures. The final extended graph is obtained from the projections after removal of scale effects and normalization, for example, with values in the unit interval [0,1]. As the user engages with the mobile app over time, the bipartite graph BG evolves into its extended graph representation. The extended graph is a graphical model of the user that, in one embodiment, is used as a building block for a recommender system.

Recommender System:

The state vector of the model is built after the model described above is generated for the current reported state at time t, which is [t, “how he/she feels”, “how he/she wants to feel”] for the case of the REMIX mobile app. The state vector of the model consists of a vector with the order labels XY and is filled with zeros in all entries except with “1” in the reported “How he/she want to feel”. The state vector sets the values of the diagonal matrix representation of the graphical model (see FIG. 2).

Given the model and state vector, the recommender system calculates the n-diffusion closure distance as follows, which is also described in Simas et al., “Distance Closures on Complex Networks,” Network Science, Vol. 6, No. 13, Dec. 12, 2013, pp. 227-268, DOI:10.1017/nws.2015.11).

The n-diffusion closure distance of E_{BG} is determined using the normalized extended graph E_{BG} of FIG. 10, where the weights represent the degree of similarity. That is, weights closer to 1.0 are more similar, whereas weights closer to 0.0 are more dissimilar. In order to convert the normalized extended graph E_{BG} into the graph of the n-diffusion closure distance DE_{BG}, all weighted entries wᵢⱼ of E_{BG} are converted into dᵢⱼ according to:

$${d}_{ij} = φ ( {w}_{ij} ) = \frac{1}{{w}_{i j}} - 1. \qquad ( Equation 3 )$$

The dot product of the two vectors is defined by:

$${v}_{i} \cdot {v}_{j} = {\sum }_{k}^{⊕} {v}_{i k} ⊗ {v}_{j k} , where \\ x ⊕ y = \{ \begin{matrix}\frac{xy}{x + y} x , y ∈ ] 0 , 1 [ \\ y or {x}_{,} if x = + ∞ or y = + ∞ \\ 0 , if x = 0 or y = 0\end{matrix} , ∀ x , y ∈ [ 0 , + ∞ ] \\ x ⊗ y = x + y , ∀ x , y ∈ [ 0 , + ∞ ] \qquad$$

The n-diffusion closure distance of E_{BG} is then the n-dot product of E_{BG} with itself n-times:

DE_{BG}²=DE_{BG}·DE_{BG}

DE_{BG}ⁿ=DE_{BG}·DE_{BG}ⁿ⁻¹

The value of n is selected as the value of the diameter of the extended graph, which is used as an estimate of the average shortest path length according to the formula n˜log(#nodes) for a random graph.

This calculation represents the state vector sets, i.e., the set of sources and sinks in the diffusion process of the extended graph. Each sink represents the goal or reward to be obtained, whereas the set of sources represents all possible initial positions. The n-diffusion closure distance determines a diffusion path between any source to the sink. The reported “How he/she feels” defines a position in the path. The distance between “How he/she feels” and “how he/she wants to feel” provides a score on how attainable it is to achieve the reward (sink) from the initial position. The distance also indicates the effectiveness of following a set of policies in order to reach the reward.

A more detailed explanation of the determination of the n-diffusion closure distance is described in the example below. For the model of FIG. 2 showing an extended graph for a specific user at a given time t, the user reports [t, “bored”, “productive” ], which means that at time t, the user feels “bored” but wants to feel “productive”. Now, suppose the set of X nodes are: {bored, stressed, anxious, sleepy, annoyed}, and the set of Y nodes are: {calm, happy, productive, content, relaxed, excited}. In this hypothetical example, the state vector or diagonal of the model is labeled as: State_vector=[bored, stressed, anxious, sleepy, annoyed, calm, happy, productive, content, relaxed, excited]

Therefore, the state_vector is set with all zeros except for the entry “productive” (How he/she wants to feel, as reported at given time t). In addition:

1. Use a normalization factor (all zeros): norm_vector=[0,0,0,0,0,0,0,0,0,0,0] and apply the n-diffusion, as described above, and store the norm_memory (diagonal of the resulting n-diffusion), which corresponds to a scenario where there is no sink, and therefore the diffusion process represents a random walker in this graphical environment;

2. Set a state_vector=[0,0,0,0,0,0,0,1,0,0,0];

3. Set the diagonal of the user model with the state vector;

4. Calculate the distance n-diffusion;

5. Extract the diagonal vector (memory) from the distance n-diffusion; then divide the vector pointwise with the norm_vector (memory), and sort the results in ascending order.

6. “Productive” appears at the top, which is the set reward (sink), followed by the other labels of the state vector.

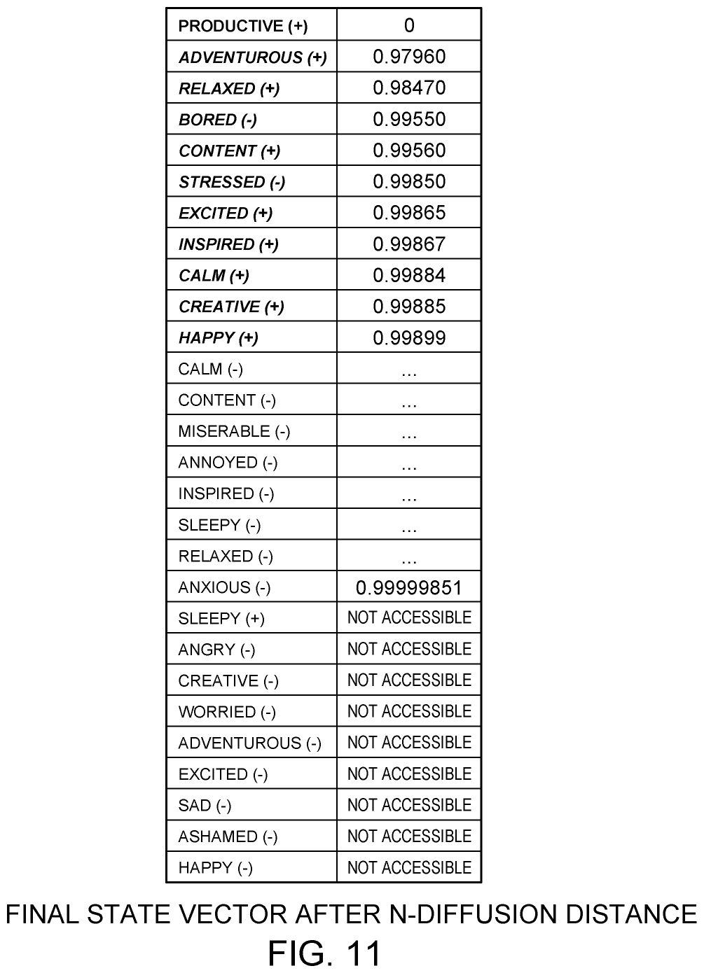

The table of FIG. 11 shows the n-diffusion ordered vector obtained from a user of the mobile app REMIX when he/she wants to be productive. The first column lists the nodes of the labeled vector, and the second column lists the diffusion distance to the sink “Productive”. The order of the listed vectors represents the shortest diffusion path between any source to the sink (“Productive”). In the table of FIG. 11, the meaning of (−) and (+) is: (−) current feeling (I feel) ‘X’; and (+) the target feeling (want to feel) ‘Y’. That is, happy (+) means that the user comes from a ‘X’ feeling (I feel) and wants to feel happy ‘Y’, and happy (−) means that the user comes from ‘X’ feeling happy (I feel happy) and wants to change this feeling to another (want to feel) ‘Y’.

The values of the n-diffusion distance that are higher than a certain threshold are considered to be random. As a non-limiting example, that threshold is 0.999 in the table of FIG. 11. In other words, the nodes (feelings) whose diffusion distance value is lower than the threshold are not considered to be random and are shown in bold letters in the table of FIG. 11. The nodes (feelings) whose diffusion distance value is higher that the threshold are considered to be random (or “trapped”) as they hardly have access to the desired feeling, in this case to be Productive (+).

In this example, it is observed that the feelings “relaxed(+)” and “adventurous(+)” are positioned between “bored(−)” and “productive(+)”, which means that when the user is “bored(−)” but tries to be “relaxed(+)” or “adventurous(+)”, the user tends towards being “productive”. From the diffusion path of the table of FIG. 11, it can be seen that the best chance for the user to become “productive” is from being “happy(+)”. “Happy(+)” as explained above is the last state through which the target “Productive(+)” can be reached that is above the threshold. All of the other feeling states that are positioned below “happy(+)” do not lead to “Productive(+)” because the diffusion process from those is trapped. The diffusion path from those other feeling states does not reach (has no access to) the selected sink “Productive(+)”, or the diffusion likelihood into the sink is very low. In addition, the feeling constructs between “happy(+)” and “productive(+)” provide the information of the policies that follow. That is, these constructs indicate the type of recommendations (in this case activities) should be recommended, which are activities that result in the feelings positioned between “happy(+)” and “productive(+).

For the particular user of this example, it would be particularly difficult to achieve a “productive” state if the user exhibits one of the constructs at the bottom of the table from “sleepy(+)” to “happy(−)”, which is intuitively the case. That is, after obtaining the diffusion path from the model and the initial state, the recommender system recommends a scored list of activities associated with constructs between “productive(+)” and “happy(+)”. In the specific example of FIG. 11, it is known that the user is X=“bored(−)”, which is in the reachable list between “Happy(+)” and Y=“Productive(+)”. Moreover, in between X=“bored(−)” and Y=“Productive(+)” are “relaxed(+)” and “adventurous(+)”. Therefore, the app recommends activities that first prime the user into a “Relaxed(+)” state followed by other activities that prime the user into an “Adventurous(+)” state in order to achieve Y=“Productive”.

Data obtained from the mobile app (such as REMIX) is used to generate a personalized graphical model. Then, after setting the target goals (sinks in the diffusion process) and determining the shortest diffusion path between the possible sources and sinks, the n-diffusion closure distance is calculated based on the user's initial state as reported or as identified based on the variables X, Y (see above). Given the user's initial state, the current position of the user along the path to the target goal is determined and the policies that are between the current position and target goal. With this information, the recommender system selects the activities that best match the policies that are in between the initial state and the target goal.

Depending on the embodiment, the modules, electronic means, and processors the perform the disclose methods are in the same or different physical locations or devices. In one embodiment, all modules are located in the same device, for example, in a central or remote server or in an electronic device of the user. In another embodiment, the modules reside in multiple servers and not in the mobile communications device itself. In yet another embodiment, some or all of the modules reside in the user's electronic device, and the rest, if any, reside in one or more servers. In the case in which at least some of the modules are in a server, the user electronic device communicates with the server through a communications network, such as a wireless communications network, a mobile communications network as 2G, 3G, 4G or LTE, LAN or W-LAN or any other type of communication networks.

Although the present invention has been described with reference to specific embodiments, it should be understood by those skilled in the art that the foregoing and various other changes, omissions and additions in the form and detail thereof may be made therein without departing from the scope of the invention as defined by the claims. Furthermore, all examples disclosed herein are intended to be only for pedagogical purposes to aid the reader in understanding the principles of the invention and the concepts contributed by the inventors to furthering the art, and are to be construed as being without limitation to such specifically disclosed examples and conditions. Moreover, all statements herein disclosing principles, aspects, and embodiments of the invention, as well as specific examples thereof, are intended to encompass equivalents thereof. Any flow charts, flow diagrams, state transition diagrams, pseudo code, and the like disclosed herein represent various processes which may be substantially represented in computer readable medium and so executed by a computer or processor, whether or not such computer or processor is explicitly shown.

1-15. (canceled)

16. A method for efficiently extracting user data and improving a well-being state of a user, the method comprising:
(a) receiving the user data onto a server, wherein the user data is acquired through interactions of the user with an application running on an electronic device of the user, wherein the user data relates to interactions that occurred over a first period of time;
(b) generating a graphical model for the user based on the user data, wherein the graphical model has nodes whose magnitudes correspond to values of the user data; and
(c) determining a recommended range of the values of the user data based on state vectors and diffusion metrics derived from the graphical model for the user, wherein the values rank activities that the application recommends to the user.

17. The method of claim 16, wherein the electronic device transmits the user data to the server.

18. The method of claim 16, wherein the electronic device is selected from the group consisting of: a laptop, a tablet, a personal computer, a portable computer, a mobile phone, a smartphone and a smartwatch.

19. The method of claim 16, wherein the generating the graphical model involves updating the graphical model based on new user data acquired during a later period of time through the application running on the electronic device of the user.

20. The method of claim 16, wherein the user data is a temporal ordered list of pairs of values of a personal construct X and an opposite personal construct Y of the user corresponding to the first period of time, and wherein the graphical model for the user includes a bipartite graph indicative of how frequently specific pairs of personal constructs and opposite personal constructs (X,Y) appear in the list.

21. The method of claim 20, wherein the graphical model for the user includes an extended graph that includes a projection of the bipartite graph to the personal construct nodes X and a projection of the bipartite graph to the opposite personal construct nodes Y, and wherein the projections are generated using a similarity measure.

22. The method of claim 21, wherein the similarity measure used is a fuzzy Jaccard similarity measure.

23. The method of claim 21, wherein the extended graph includes a transpose of the bipartite graph.

24. The method of claim 21, wherein a z-score technique is used to compensate for different scales of the bipartite graph and the projections that are included together in the extended graph.

25. The method of claim 20, wherein the personal construct X represents a current state of the user, and wherein the opposite personal construct Y represents a desired state of the user.

26. The method of claim 16, wherein the determining the recommended range of the values of the user data further comprises:
(c1) determining an initial state of the user and a target goal of the well-being state of the user;
(c2) based on the generated graphical model, the initial state, and the target goal, calculating an n-diffusion closure distance to determine a shortest diffusion path between the initial state and the target goal; and
(c3) based on the determined shortest diffusion path, determining a recommended range of values that rank activities to be undertaken by the user in order to reach the target goal from the initial state.

27. The method of claim 16, wherein the ranked activities are recommended to the user through a user interface of the electronic device of the user.

28. A system for efficiently extracting user data and improving a well-being state of a user, comprising:
a database that stores user data, wherein the user data is acquired through interactions of the user with an application running on an electronic device of the user, wherein the user data relates to interactions that occurred over a first period of time; and
a processing unit of the electronic device of the user adapted to execute program instructions of the application, wherein the program instructions when executed cause the electronic device to:
retrieve the user data from the database;
generate a graphical model for the user based on the user data, wherein the graphical model has nodes whose magnitudes correspond to values of the user data;
determine a recommended range of the values of the user data based on state vectors and diffusion metrics derived from the graphical model for the user, wherein the values rank activities that the application recommends to the user; and
recommend that the user engage in one of the ranked activities.

29. The system of claim 28, wherein the ranked activities are recommended to the user through a user interface of the electronic device of the user.

30. The system of claim 28, wherein the electronic device is selected from the group consisting of: a laptop, a tablet, a personal computer, a portable computer, a mobile phone, a smartphone and a smartwatch.

31. The system of claim 28, wherein the graphical model is generated by updating the graphical model based on new user data acquired during a later period of time through the application running on the electronic device of the user.

32. The system of claim 28, wherein the user data is a temporal ordered list of pairs of values of a personal construct X and an opposite personal construct Y of the user corresponding to the first period of time, and wherein the graphical model for the user includes a bipartite graph indicative of how frequently specific pairs of personal constructs and opposite personal constructs (X,Y) appear in the list.

33. The system of claim 32, wherein the personal construct X represents a current state of the user, and wherein the opposite personal construct Y represents a desired state of the user.

34. The system of claim 32, wherein the graphical model for the user includes an extended graph that includes a projection of the bipartite graph to the personal construct nodes X and a projection of the bipartite graph to the opposite personal construct nodes Y.

35. The system of claim 34, wherein the extended graph includes a transpose of the bipartite graph.
